(19) United States
(12) Patent Application Publication
(10) Pub. No.: US 2018/0214593 A1
(43) Pub. Date: Aug. 2, 2018

(54) SALT LAMP DIFFUSER

(71) Applicant: Believe It LLC, Savannah, NY (US)

(72) Inventors: Ryan T. Breen, Savannah, NY (US); Gilles Beeckmans, Brussels (BE)

(21) Appl. No.: 15/419,824

(22) Filed: Jan. 30, 2017

(51) Int. Cl. A61L 9/12; A61L 9/03; B05B 17/06; B05B 9/04; F21V 33/00

(52) U.S. Cl. CPC A61L 9/12; A61L 9/03; B05B 17/0607; A61L 2209/135; F21V 33/0064; A61L 2209/132; B05B 9/04

(57) ABSTRACT

A system for diffusing a composition includes a base that defines a first opening and a second opening and which encloses a space, the space comprising at least a portion of a composition reservoir and a diffusing element. The composition reservoir may be associated with the first opening and configured to receive the composition at the first opening. The composition reservoir may also comprise a sloped wall associated with a diffusing element, the diffusing element configured to diffuse the composition at the second opening. The diffuser system may further comprise a salt formation.

### BACKGROUND

### 1. Technical Field

This disclosure generally relates to diffusers. More specifically, the present disclosure relates to salt lamp diffusers.

### 2. Related Technology

Diffusers may be used for aromatherapy. Aromatherapy is the practice of using the natural oils extracted from flowers, bark, stems, leaves, roots or other parts of a plant to enhance an individual's psychological and physical well-being. Aromatherapy, as indicated by its name, enhances psychological and physical well-being through smell.

Many practitioners of this art routinely employ a variety of natural oil extracts to enhance or otherwise affect either their own or another person's physiological and physical well-being. Oils, combinations of oils, and combinations of oils with water or other material can be used to decrease stress, increase productivity, or promote clarity of thought. In some circumstances, the oils are applied directly to the skin of the person who desires their beneficial effects. In other instances, a diffuser is used to diffuse the oils into the environment surrounding the person who desires their beneficial effects. Thus, diffusers can enhance an individual's psychological and physical well-being.

Like diffusers, salt lamps can enhance an individual's psychological and physical well-being. Salt lamps generate negative ions by attracting moisture and/or water from the air to the surface of a salt formation. The moisture and/or water then quickly evaporates as a result of heat from a heating element in the lamp. These negative ions cleanse the air and can neutralize and restore air quality.

However, combining the beneficial effects of a salt lamp with the beneficial effects of a diffuser can be difficult for various reasons. For example, the material diffused by the diffuser may collect on the salt formation of the salt lamp, causing the salt formation to erode and deteriorate. Such erosion and deterioration can degrade the aesthetic qualities of the salt lamp while the salt formation is still functional and may reduce the working life of the salt formation and therefore the salt lamp. Erosion and deterioration may also be a safety hazard because the degradation of the salt formation may create harmful chemical reactions because salt may evaporate from the inner surface of the salt formation and mix with the mechanical and electrical components of the diffuser system.

In addition, combining a salt lamp with a diffuser may be difficult because the presence of the heated element used to heat the salt formation may preclude inclusion of a diffuser. For example, the heating element may unnecessarily warm the material to be diffused because the heating element may reside spatially too close to the material to be diffused. Alternatively, the heating element may cause the material to be diffused to evaporate within the body of the diffuser and aggregate on the inner wall of the salt formation. This aggregation can cause the salt lamp to corrode and deteriorate and may harm the aesthetic qualities of the diffuser.

In addition, combining a salt lamp with a diffuser may be difficult because it may preclude the necessary angling of the reservoir housing the material to be diffused. Without the proper angling of that reservoir, the material to be diffused may become stagnant and stale.

Accordingly, there are a number of disadvantages with diffusers that can be addressed.

### BRIEF SUMMARY

Implementations of the present disclosure solve one or more of the foregoing or other problems in the art with systems and apparatuses for a salt lamp diffuser. In particular, one or more implementations can include a diffuser system having a base that defines a first opening and a second opening and which encloses a space, the space comprising at least a portion of a composition reservoir and a diffusing element. The composition reservoir may be associated with the first opening and configured to receive the composition at the first opening. The composition reservoir may also comprise a sloped wall associated with a diffusing element, the diffusing element being configured to diffuse the composition at the second opening. The diffuser system may further comprise a salt formation.

Diffuser systems of the present disclosure can also include a frequency switch and intensity dial. The diffuser system may further include a connector for communicating with a power source.

This summary is provided to introduce a selection of concepts in a simplified form that are further described below in the Detailed Description. This Summary is not intended to identify key features or essential features of the claimed subject matter, nor is it intended to be used as an indication of the scope of the claimed subject matter.

Additional features and advantages of the disclosure will be set forth in the description which follows, and in part will be obvious from the description, or may be learned by the practice of the disclosure. The features and advantages of the disclosure may be realized and obtained by means of the instruments and combinations particularly pointed out in the appended claims. These and other features of the present disclosure will become more fully apparent from the following description and appended claims, or may be learned by the practice of the disclosure as set forth hereinafter.

### BRIEF DESCRIPTION OF THE DRAWINGS

In order to describe the manner in which the above recited and other advantages and features of the disclosure can be obtained, a more particular description of the disclosure briefly described above will be rendered by reference to specific embodiments thereof, which are illustrated in the appended drawings. It is appreciated that these drawings depict only typical embodiments of the disclosure and are not therefore to be considered to be limiting of its scope. The disclosure will be described and explained with additional specificity and detail through the use of the accompanying drawings in which:

FIG. 1 illustrates a perspective view of a diffuser system according to one or more implementations of the present disclosure.

FIG. 2 illustrates an elevational view of the diffuser system of FIG. 1.

FIG. 3 illustrates a perspective view of the base of the diffuser system of FIGS. 1 and 2.

FIG. 4 illustrates another perspective view of the base of FIG. 3.

FIG. 5 illustrates a top plan view of the base of FIG. 3.

FIG. 6 illustrates a cross-sectional view of a portion of the base of FIG. 3.

FIG. 7 illustrates an elevational view of the base of FIG. 3.

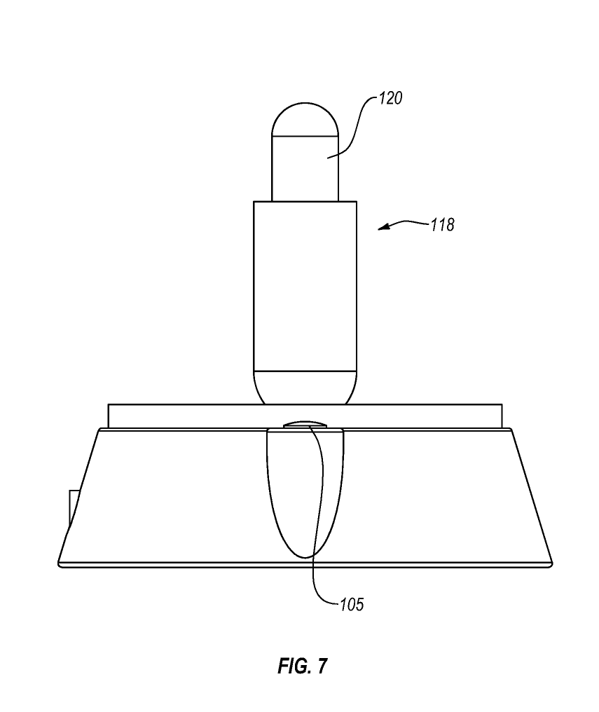

FIG. 8 illustrates internal elements of the diffuser system illustrated in FIGS. 1-6 according to one or more implementations of the present disclosure.

FIG. 9 illustrates a bottom perspective view of the salt lamp diffuser system of FIGS. 1-8 according to one or more implementations of the present disclosure.

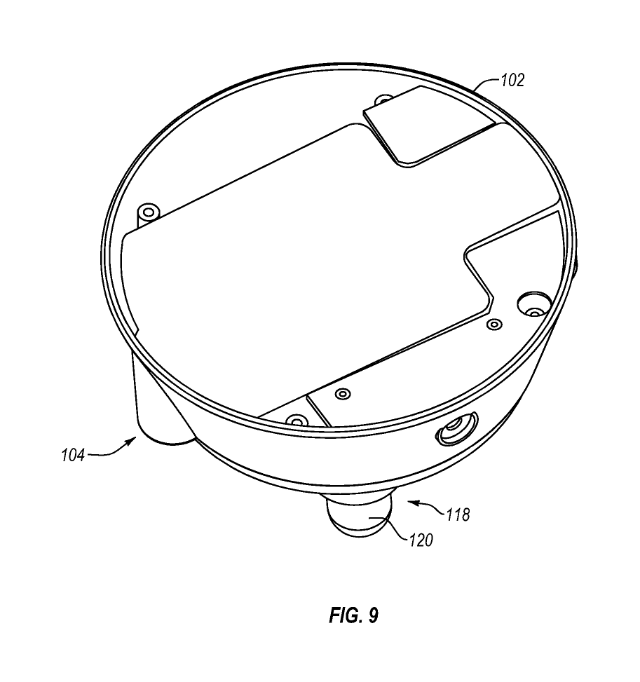

### DETAILED DESCRIPTION

Before describing various embodiments of the present disclosure in detail, it is to be understood that this disclosure is not limited to the parameters of the particularly exemplified systems, methods, apparatus, products, processes, and/or kits, which may, of course, vary. Thus, while certain embodiments of the present disclosure will be described in detail, with reference to specific configurations, parameters, components, elements, etc., the descriptions are illustrative and are not to be construed as limiting the scope of the claimed invention. In addition, the terminology used herein is for the purpose of describing the embodiments, and is not necessarily intended to limit the scope of the claimed invention.

Unless defined otherwise, all technical and scientific terms used herein have the same meaning as commonly understood by one of ordinary skill in the art to which the present disclosure pertains.

Various aspects of the present disclosure, including devices, systems, and methods may be illustrated with reference to one or more embodiments or implementations, which are exemplary in nature. As used herein, the term “exemplary” means “serving as an example, instance, or illustration,” and should not necessarily be construed as preferred or advantageous over other embodiments disclosed herein. In addition, reference to an “implementation” of the present disclosure or invention includes a specific reference to one or more embodiments thereof, and vice versa, and is intended to provide illustrative examples without limiting the scope of the invention, which is indicated by the appended claims rather than by the following description.

As used throughout this application the words “can” and “may” are used in a permissive sense (i.e., meaning having the potential to), rather than the mandatory sense (i.e., meaning must). Additionally, the terms “including,” “having,” “involving,” “containing,” “characterized by,” as well as variants thereof (e.g., “includes,” “has,” and “involves,” “contains,” etc.), and similar terms as used herein, including the claims, shall be inclusive and/or open-ended, shall have the same meaning as the word “comprising” and variants thereof (e.g., “comprise” and “comprises”), and do not exclude additional, un-recited elements or method steps, illustratively.

It will be noted that, as used in this specification and the appended claims, the singular forms “a,” “an” and “the” include plural referents unless the context clearly dictates otherwise. Thus, for example, reference to a “salt formation” includes one, two, or more salt formations. Similarly, reference to a plurality of referents should be interpreted as comprising a single referent and/or a plurality of referents unless the content and/or context clearly dictate otherwise. Thus, reference to “salt formations” does not necessarily require a plurality of such salt formations. Instead, it will be appreciated that independent of conjugation; one or more salt formations are contemplated herein.

As used herein, directional terms, such as “top,” “bottom,” “left,” “right,” “up,” “down,” “upper,” “lower,” “proximal,” “distal” and the like are used herein solely to indicate relative directions and are not otherwise intended to limit the scope of the disclosure and/or claimed invention.

Various aspects of the present disclosure can be illustrated by describing components that are bound, coupled, attached, connected, and/or joined together. As used herein, the terms “bound,” “coupled”, “attached”, “connected,” and/or “joined” are used to indicate either a direct association between two components or, where appropriate, an indirect association with one another through intervening or intermediate components. In contrast, when a component is referred to as being “directly bound,” “directly coupled”, “directly attached”, “directly connected,” and/or “directly joined” to another component, no intervening elements are present or contemplated. Furthermore, binding, coupling, attaching, connecting, and/or joining can comprise mechanical and/or chemical association.

To facilitate understanding, like reference numerals (i.e., like numbering of components and/or elements) have been used, where possible, to designate like elements common to the figures. Specifically, in the exemplary embodiments illustrated in the figures, like structures, or structures with like functions, will be provided with similar reference designations, where possible. Specific language will be used herein to describe the exemplary embodiments. Nevertheless it will be understood that no limitation of the scope of the disclosure is thereby intended. Rather, it is to be understood that the language used to describe the exemplary embodiments is illustrative only and is not to be construed as limiting the scope of the disclosure (unless such language is expressly described herein as essential).

The headings used herein are for organizational purposes only and are not meant to be used to limit the scope of the description or the claims.

The present disclosure extends to systems and apparatuses for diffusing a composition. In particular, one or more implementations include a diffuser system having a base that defines a first opening and a second opening and which encloses a space, the space comprising at least a portion of a composition reservoir and a diffusing element. The composition reservoir may be associated with the first opening and configured to receive the composition at the first opening. The composition reservoir may also comprise a sloped wall associated with a diffusing element, the diffusing element being configured to diffuse the composition at the second opening. The diffuser system may further comprise a salt formation.

In some embodiments of the present disclosure, the diffuser receives and retains a diffusible composition such as, for example, a composition of one or more oils. The composition may include a volume of water or other fluid combined with one or more oils that when diffused creates aromatherapeutic effects.

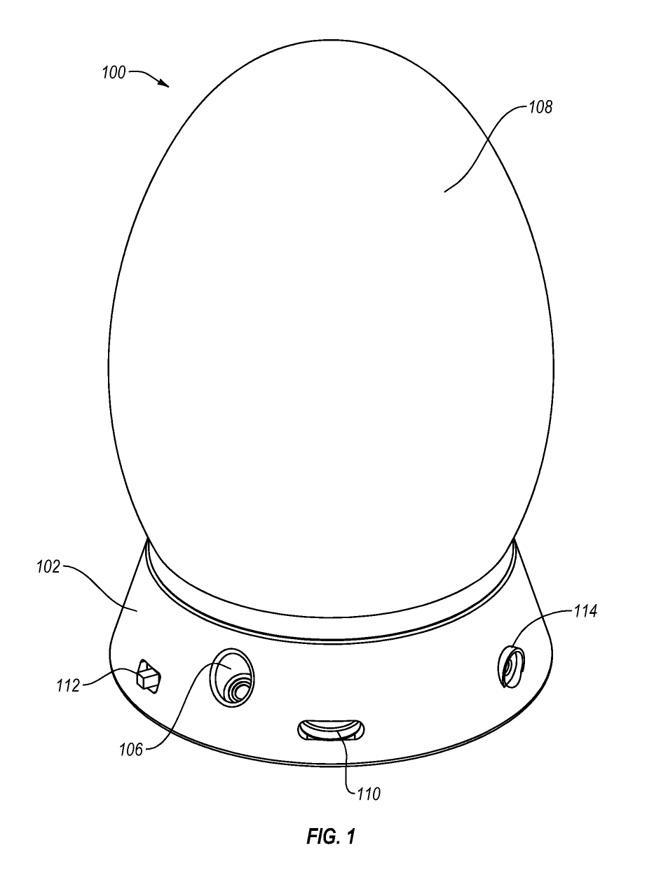

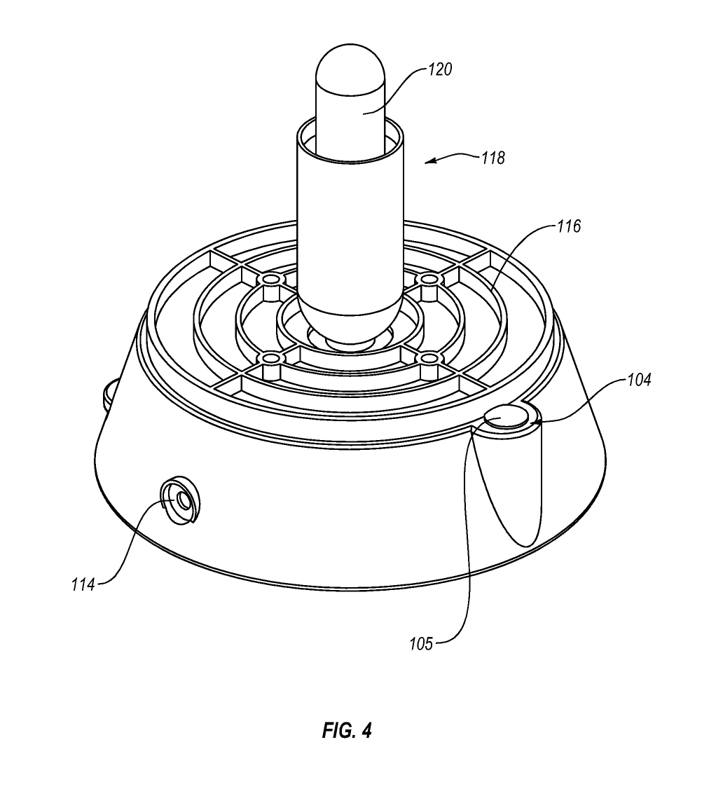

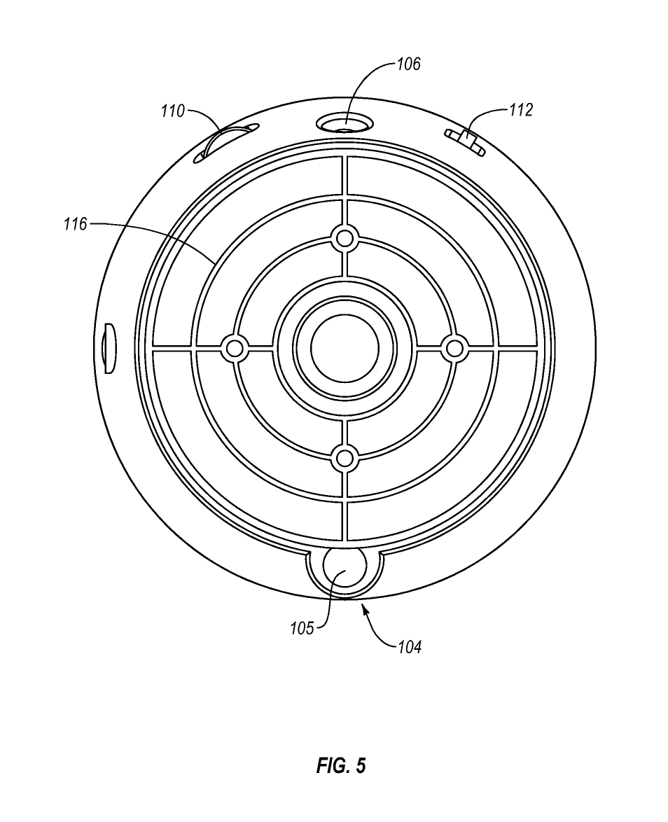

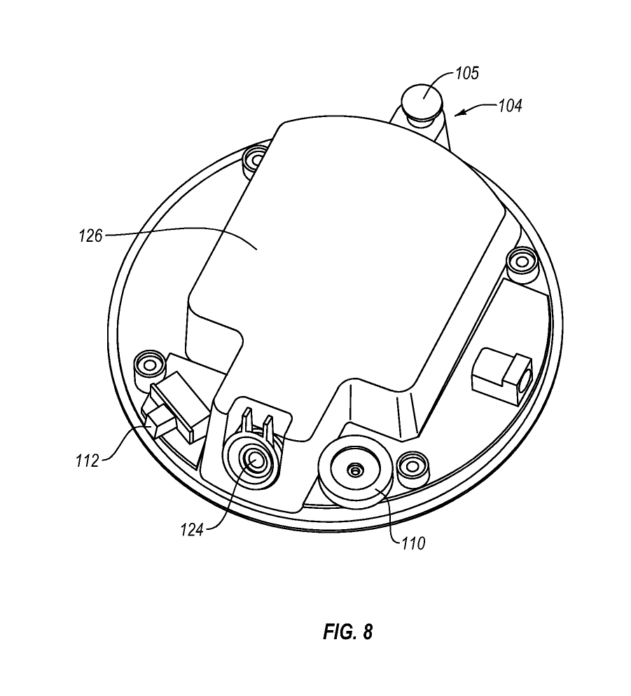

Reference will now be made to the figures of the present disclosure. For example, FIG. 1 illustrate a perspective view of a diffuser system according to one or more implementations of the present disclosure. The diffuser 100 comprises a base 102 defining a first opening 104 (FIGS. 4-6, 8), a second opening 106, an intensity dial 110, and a frequency switch 112.

The base may, as depicted in FIG. 1, be circular in shape, having a flat bottom surface. However it should be understood that base 102 may be any shape, such as spherical, ovoid, prismatic, oblong, polygonal, or any combination and/or variation of the foregoing. In certain embodiments the circular shape of base 102 may provide the benefits of being ergonomic and/or easy to handle. In addition, the flat bottom surface of base 102 may promote the stability of the diffuser 100 when in a prone position and may additionally encourage anything placed within the diffuser 100 (e.g., the diffusible composition) to rest within the diffuser 100 and not leak out of the diffuser 100 (e.g., through an unobstructed first opening).

As noted previously, it should be appreciated that the base 102 and/or contouring of the diffuser is not limited to what is depicted in the accompanying figures. Rather, the diffuser 100 may be of any shape and may include any contour. For example, the diffuser 100 may be spherical, ovoid, prismatic, oblong, polygonal, or any combination and/or variation of the foregoing. Further, although the first and second openings 104, 106 defined by the base 102 are depicted as being substantially circular, the fist and second openings 104, 106 may be of any shape and of any size and may be defined at any location on the base 102. The base 102 may comprise any suitable material, including without limitation plastics, thermoplastics, thermoplastic elastomers, elastomers, rubbers, bamboo, woods, glass, or combinations thereof.

The first opening 104 may in some embodiments, and as depicted in FIGS. 4-6 and 8, be defined by the base 102 such that the first opening 104 is sized and shaped to receive an obturation 105. The obturation 105 may be removably attached to the first opening 104 such that the obturation 105 substantially seals and/or covers the first opening 104 when attached to the first opening 104, or the obturation 105 may be removed from the first opening 104 such that the first opening 104 is substantially unobstructed.

The obturation 105 may comprise a first element and a second element wherein the first element protrudes within the first opening 104 when the obturation 105 is attached thereto and the second element rests on the body of the diffuser. The obturation 105 may be of any shape and/or size, which may be the same or different than the shape and/or size of the first opening 104. The obturation 105 may comprise any material, including without limitation, elastomers, thermoplastic elastomers, rubber, wood, cork, plastics, thermoplastics, and combinations thereof

In some embodiments, the obturation 105 may include only the second portion as described above. In such an embodiment, the obturation 105 may not protrude within the first opening 104, but may rest on top of the second opening 106. The obturation 105 may have an adhesive surface facing the base 102 that adheres the obturation 105 to the portion of the base 102 defining the first opening 104, thereby serving a similar function as one or more of the foregoing obturations by sealing the first opening 104. The obturation 105 may also connect or attach to the base 102 in any other fashion in such a way as to server a similar function as one or more of the foregoing obturations by sealing the first opening 104.

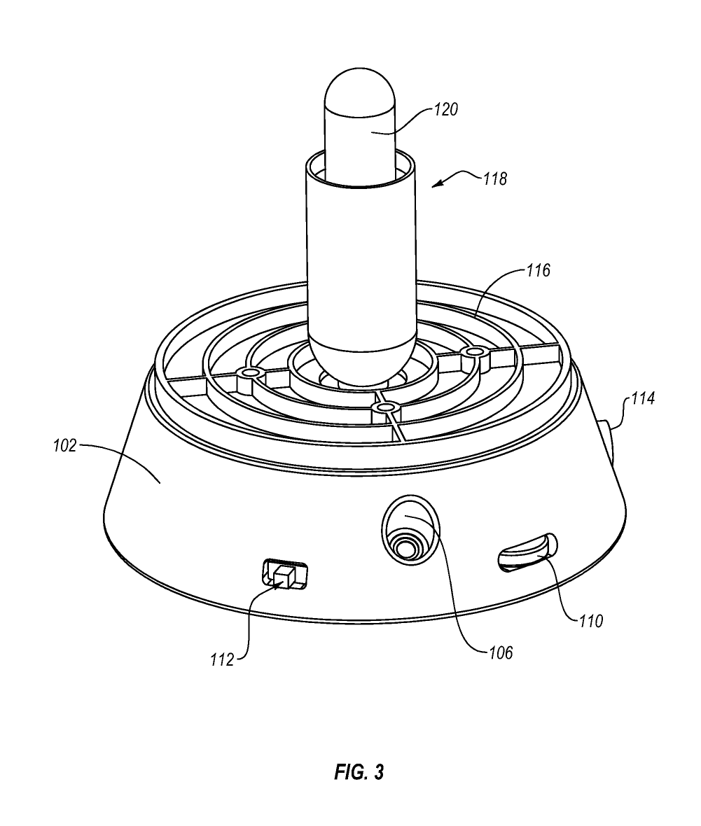

As noted above, the diffuser 100 may further include an intensity dial 110. As shown in FIG. 1, intensity dial 110 may be a circular dial that may be adjusted by the user by rotating the dial to the right of the left. Intensity dial 110 may be connected to a heating element 118 (FIG. 3) such that, when rotated, intensity dial 110 causes heating element 118 to increase or decrease in luminosity and heat. For example, rotating intensity dial 110 to the right may increase the luminosity and heat expelled by heating element 118 while rotating intensity dial 110 to the left may decrease the luminosity and heat expelled by heating element 118.

As also noted above, the salt lamp diffuser 100 may further include a frequency switch 112. As shown in FIG. 1, frequency switch 112 may be a rectangular switch that may be switched to any of multiple (e.g., two, three, or more) frequency positions. As one illustrative example, when frequency switch 112 is in a first (e.g., left) frequency position, the salt lamp diffuser 100 may emit essential oils, or any other material to be diffused, every 15 seconds. Conversely, when frequency switch 112 is in a second (e.g., middle) frequency position, the salt lamp diffuser 100 may emit essential oils, or any other material to be diffused, every 30 seconds. Similarly, when frequency switch 112 is in a third (e.g., right) frequency position, the salt lamp diffuser 100 may emit essential oils, or any other material to be diffused, every 60 seconds. One of skill in the art will understand that the frequencies listed herein for the various frequency positions are only illustrative and each frequency position may have differing frequencies than those listed. In addition, all or some of the frequency positions may correspond to the same frequency rather than different frequencies.

While FIG. 1 depicts intensity dial 110 and frequency switch 112 as circular and rectangular shapes, respectively, and as moving on horizontal planes, it should be understood that intensity dial 110 and frequency switch 112 may be of any shape and move in any plane. In some embodiments, the diffuser 100 may have a light which illuminates intensity dial 110 and/or frequency switch 112 when the connector 114 (described below) is connected to a power source with sufficient power to run the salt lamp diffuser 100. Further, frequency switch 110 may have two frequency positions, three frequency positions, four frequency positions, five frequency positions, six frequency positions, seven frequency positions, eight frequency positions, nine frequency positions, ten frequency positions, or any number of frequency positions that may variably adjust the rate and/or volume of composition diffused by the device. For example, the diffuser may have three frequency positions that adjust the frequency (e.g., every two minutes, every four minutes, and every six minutes) by which a specified volume of composition is diffused. The differences, timing, and/or volume of composition diffused at each setting may be preset or may be set by the user. As a further example, the diffuser may have an adjustment switch operable by a keypad that the user may input an exact time interval and/or volume of composition to be diffused.

The diffuser 100 may further include a connector 114 for communicating with a power source. The connector 114 may connect to and/or draw power from a power source, such as, for example, an AC power cord having a complementary connector port. As a non-limiting example, the connector 114 associated with the salt lamp diffuser 100 may connect through an AC power cord to a wall jack in a home or office and draw power from the home or office to run one or more elements of the diffuser 100 (e.g., the diffusing element 116, the intensity dial 110, the frequency switch 112, etc.). The connector may be any connector that is configured to connect the diffuser 100 to a power source having sufficient power to run one or more elements of the diffuser. In addition to the power source of a home or business, exemplary power sources include a laptop computer, a desktop computer, a mobile phone, a digital music player, and similar electronic and/or computing devices.

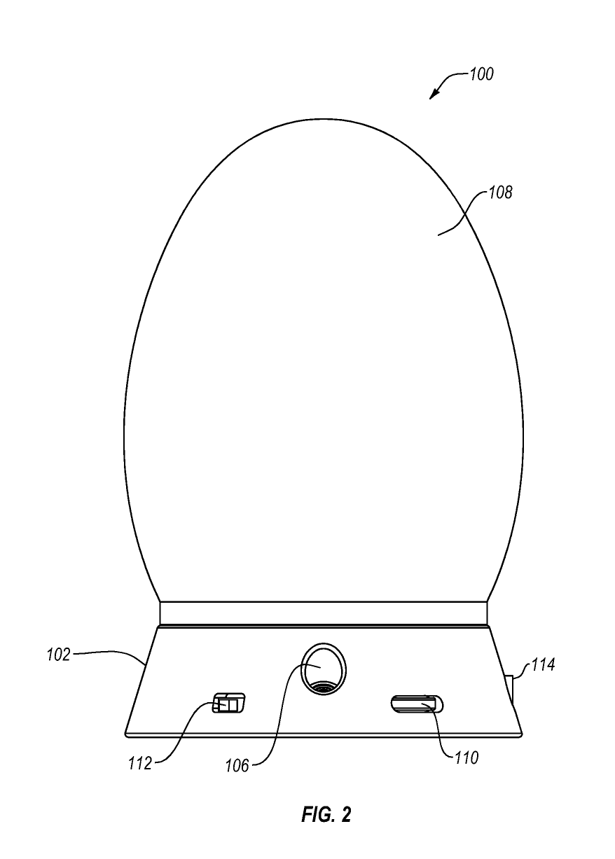

The salt lamp diffuser 100 may include a salt formation 108, as shown in FIGS. 1 and 2. Salt formation 108 may consist of mined salt, such as Himalayan salt, Russian salt, or European salt, which has been hollowed out to allow room for heating element 118 to protrude into the middle of the hollowed out salt formation 108. Although salt formation 108 may consist of a salt from a specific geographic region as indicated above, it also may consist of any other type of salt. Alternatively, salt formation 108 may consist of a mineral other than salt. Salt formation 108 may be pinkish orange in color due to the high concentration of minerals within the formation.

When heated, salt formation 108 may release negative ions which may have positive physical and psychological effects on users of the diffuser 100. For example, office workers who suffer health complaints around fluorescent lighting and computer monitors may experience less fatigue and headaches if the concentration of negative ions in the air is increased. As another example, negative ions expelled by salt formation 108 may bind with airborne pollutants, making them heavier so that they fall to the ground and are unavailable to be inhaled. As another example, an increase in negative ions in the air may increase blood flow to the brain and improve concentration.

As shown in FIG. 1, the salt formation 108 may join with base 102 to create the salt lamp diffuser 100. Salt formation 108 may join with base 102 through any type of connection or through no connection at all. For example, salt formation 108 may be adhered to base 102 using glue, tape, hook and loop fasteners, or any other suitable fastener. Alternatively, salt formation 108 and base 102 may be configured such that salt formation 108 screws onto base 102, or base 102 screws onto salt formation 108. Alternatively, salt formation 108 may not connect in any permanent way to base 102 but may instead simply rest on base 102. When combined, the salt formation 108 may cover the top of base 102 such that the top of base 102 is not visible. Alternatively, salt formation 108 may only cover a portion of the top of base 102 leaving some of the top of base 102 visible.

Looking now at FIGS. 3-5, the top of base 102 is shown without salt formation 108 connected thereto. The top of base 102 may include a separator 116 and heating element 118. Separator 116 may comprise any material, including without limitation, elastomers, thermoplastic elastomers, rubber, wood, cork, plastics, thermoplastics, and combinations thereof. Separator 116 may have a ribbed surface as depicted or any other surface. Separator 116 may act as a support for salt formation 108 when salt formation 108 is connected to or positioned on or disposed on base 102. In addition, separator 116 may serve to separate an inner cavity of base 102 from heating element 118 and salt formation 108. Further, separator 116 may act as a barrier to prevent moisture that may build on an inner wall of salt formation 108 from contacting portions of intensity dial 110, frequency switch 112, and connector 114 that reside inside base 102. Likewise, separator 116 may act as a barrier to prevent the material to be diffused from migrating to the inner wall of salt formation 108.

Heating element 118 may include a lighting element 120. Lighting element 120 may be a single LED or a plurality of LEDs, such as one or more LED arrays. If a plurality of LEDs are used, they may be arranged in, for example, a line, a circle, a square, a flower shape, a rainbow shape, or any other desired shape or arrangement. Lighting element 120 may employ LEDs of the same or different colors. For example, red, white, and blue LEDs could be used during the Fourth of July holiday. Alternatively, lighting element 120 may be a standard 25 watt tubular bulb. In such an embodiment, lighting element 120 may be made of glass and roughly 2 inches tall so as to protrude within the hollowed out cavity of salt formation 108. However, one of skill in the art will understand that lighting element 120 may be of any height, shape, or material so as to light salt formation 108 from the interior thereof.

In one embodiment, lighting element 120 may provide sufficient light, i.e., is of a sufficient luminous intensity, to function as a nightlight. That is, lighting element 118 may have a luminous intensity of at least 1300 millicandela (mcd) at 20 milliamps (mA) or at least 5000 mcd at 20 mA. Alternatively, lighting element 118 may have a low nominal luminous intensity on the order of a few hundred mcd. Lighting element 118 may also use multiple LED arrays, each having a specific luminous intensity, either the same or different from each other. If lighting element 118 consists of multiple LED arrays they may be controlled independently or together, to provide an ornamental design.

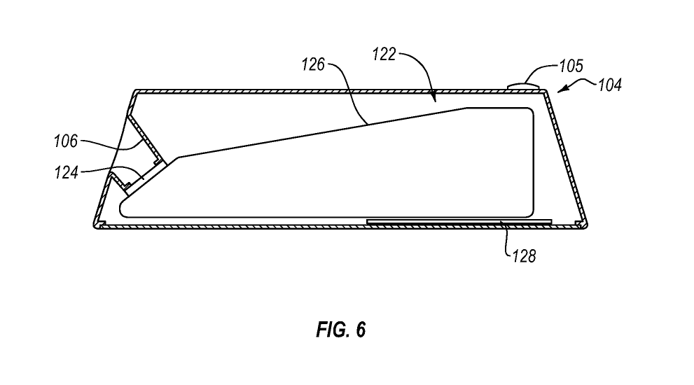

As shown in FIG. 6, the diffuser 100 may include a diffusing element 124. The diffusing element 124 may include any element known in the art for diffusing compositions, including without limitation, a piezoelectric element, a heating element, a nebulizer, or combinations thereof Such diffusing elements are well known in the art, such as that described in U.S. Pat. Nos. 6,232,129, 3,840,758, 5,518,179, 4,432,699, and 5,622,897, each of which is incorporated herein by reference.

With continued reference to FIG. 6, depicted are internal elements housed—or at least partially housed—within base 102. The forthcoming elements may be included within one or more embodiments of salt lamp diffuser 100 described above. Salt lamp diffuser 100 includes a composition reservoir 122 in communication with and/or associated with the first opening 104 and which is configured to receive one or more compositions at the first opening 104. The composition reservoir 122 (and any contents therein) may be at least partially sealed within the base 102 of salt lamp diffuser 100 when the obturation 105 is associated with the second opening 106.

As depicted, the composition reservoir 122 may comprise a sloped surface 126 with the diffusing element 124 associated therewith. In some embodiments, the sloped surface 126 allows for wickless transfer of the composition from the composition reservoir 122 to the diffusing element 124. Wickless transfer of the composition to be diffused provides many benefits, including, for example, a reduction in the cost of the diffuser and a reduction in the amount of time and money required to maintain the diffuser. Wicks typically require replacement after a period of use and can retain residue of previously used compositions, which may taint the effects of a pure natural oil composition designed for a particular purpose. Replacing a wick may be a time intensive task because it often requires dismantling the broader diffuser system. Further, a wickless diffuser has a wider range of compositions that it may diffuse compared to diffusers that use a wick. Wick-based diffusers are limited to compositions within a particular range of viscosities. If the composition is too viscous, it will not transit the wick and will not, therefore, be diffused. A wickless apparatus as described in one or more embodiments of the present disclosure overcomes these aforementioned problems.

Also illustrated in FIG. 6 is printed circuit board 128. In some embodiments, the printed circuit board 128 is in electronic communication with the diffusing element 124, the intensity dial 110, the frequency switch 112, and/or the connector 114. The printed circuit board 128 may, in some embodiments, control the timing of diffusion and/or any programmed/programmable feature associated with the diffuser 100. The printed circuit board 128 may comprise one or more components for performing any of the desired functionality as described herein and as known in the art.

One will appreciate that implementations of the present disclosure can provide a wide variety of salt lamp diffuser systems that provide a wide variety of benefits. For example, implementations of the present disclosure can provide a diffuser system having a base that defines a first opening and a second opening and which encloses a space, the space comprising at least a portion of a composition reservoir and a diffusing element. The composition reservoir may be associated with the first opening and configured to receive the composition at the first opening. The composition reservoir may also comprise a sloped wall associated with a diffusing element, the diffusing element configured to diffuse the composition at the second opening. The diffuser system may further comprise a salt formation.

Various alterations and/or modifications of the inventive features illustrated herein, and additional applications of the principles illustrated herein, which would occur to one skilled in the relevant art and having possession of this disclosure, can be made to the illustrated embodiments without departing from the spirit and scope of the invention as defined by the claims, and are to be considered within the scope of this disclosure. Thus, while various aspects and embodiments have been disclosed herein, other aspects and embodiments are contemplated. While a number of methods and components similar or equivalent to those described herein can be used to practice embodiments of the present disclosure, only certain components and methods are described herein.

It will also be appreciated that systems, devices, products, kits, methods, and/or processes, according to certain embodiments of the present disclosure may include, incorporate, or otherwise comprise properties, features (e.g., components, members, elements, parts, and/or portions) described in other embodiments disclosed and/or described herein. Accordingly, the various features of certain embodiments can be compatible with, combined with, included in, and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include said features, members, elements, parts, and/or portions without necessarily departing from the scope of the present disclosure.

Moreover, unless a feature is described as requiring another feature in combination therewith, any feature herein may be combined with any other feature of a same or different embodiment disclosed herein. Furthermore, various well-known aspects of illustrative systems, methods, apparatus, and the like are not described herein in particular detail in order to avoid obscuring aspects of the example embodiments. Such aspects are, however, also contemplated herein.

The present disclosure may be embodied in other specific forms without departing from its spirit or essential characteristics. The described embodiments are to be considered in all respects only as illustrative and not restrictive. The scope of the invention is, therefore, indicated by the appended claims rather than by the foregoing description. While certain embodiments and details have been included herein and in the attached disclosure for purposes of illustrating embodiments of the present disclosure, it will be apparent to those skilled in the art that various changes in the methods, products, devices, and apparatus disclosed herein may be made without departing from the scope of the disclosure or of the invention, which is defined in the appended claims. All changes which come within the meaning and range of equivalency of the claims are to be embraced within their scope.

What is claimed is:

1. An apparatus for diffusing a composition, the apparatus comprising:
a base defining a first opening and a second opening, the base enclosing a space that comprises at least a portion of:
a composition reservoir associated with the first opening, wherein the composition reservoir is configured to receive the composition at the first opening and wherein the composition reservoir comprises a sloped wall; and
a diffusing element associated with the sloped wall, wherein the diffusing element is configured to diffuse the composition at the second opening; and
a salt formation disposed on the base.

2. The apparatus as in claim 1, wherein at least a portion of the composition is diffused at the diffusing element after being wicklessly drawn from the composition reservoir.

3. The apparatus as in claim 1, wherein the system further comprises a frequency switch.

4. The apparatus as in claim 1, wherein the system further comprises an intensity dial.

5. The apparatus as in claim 1, further comprising an obturation that is configured to removably attach to the second opening and wherein the obturation restricts the flow of the contents of the composition reservoir from the second opening when the obturation is attached to the second opening.

6. The apparatus as in claim 1, wherein the system further comprises a connector for communicating with a power source.

7. The apparatus as in claim 1, wherein the salt formation comprises at least one of Himalayan salt, Russian salt, or European salt.

8. The apparatus as in claim 1, wherein the salt formation screws onto the base.

9. The apparatus as in claim 1, further comprising a separator disposed on one side of the base.

10. The apparatus as in claim 9, wherein the separator has a ribbed surface.

11. The apparatus as in claim 1, further comprising a heating element disposed at least partially within the salt formation.

12. The apparatus as in claim 1, further comprising a lighting element disposed at least partially within the salt formation.

13. The apparatus as in claim 1, wherein the composition reservoir is substantially housed within the base.

14. A system for diffusing a composition, the system comprising:
a base defining a first opening and a second opening;
a salt formation disposed on the base;
a composition reservoir housed within the base and associated with the first opening, wherein the composition reservoir is configured to receive the composition at the first opening and wherein the composition reservoir comprises a sloped wall;
a diffusing element associated with the sloped wall, wherein the diffusing element is configured to draw the composition from the composition reservoir and diffuse the composition at the second opening;
a printed circuit board configured to control the diffusing element;
a connector for communicating with a power source; and
an obturation configured to be removably attached to the second opening.

15. The apparatus as in claim 14, wherein the salt formation screws onto the base.

16. The apparatus as in claim 14, further comprising a heating element disposed at least partially within the salt formation.

17. The apparatus as in claim 14, further comprising a separator disposed on at least one side of the base.

18. The apparatus as in claim 14, wherein the diffusing element is configured to diffuse at least a portion of the composition after the composition is wicklessly drawn from the composition reservoir.

19. The apparatus as in claim 14, wherein the diffusing element is selected from the group consisting of: a piezoelectric element, a heating element, a nebulizer, and combinations thereof.

20. A system for diffusing a composition, the system comprising:
a base comprising a composition reservoir, one or more openings in communication with the composition reservoir, and a diffuser element for diffusing the composition through the one or more openings;
a salt formation disposed on the base; and
at least one of a lighting element or heating element that lights or heats the salt formation from an interior of the salt formation.
